(19) United States
(12) Patent Application Publication
(10) Pub. No.: US 2023/0187044 A1
(43) Pub. Date: Jun. 15, 2023

(54) SECURE SYSTEM FOR MONITORING AND DISPENSING DOSES OF ACTIVE SUBSTANCES PRE-DETERMINED BY AN EXPERT AND CUSTOMISED FOR USERS

(71) Applicant: LINKEDTECH, Paris (FR)

(72) Inventor: Adrien PLECIS, Cachan (FR)

(21) Appl. No.: 17/921,383

(22) PCT Filed: Apr. 27, 2021
(86) PCT No.: PCT/EP2021/061016

§ 371 Date: Oct. 26, 2022

(30) Foreign Application Priority Data
Apr. 28, 2020 (FR) 2004234

(51) Int. Cl. G16H 20/13; G16H 40/67

(52) U.S. Cl. CPC G16H 20/13; G16H 40/67

(57) ABSTRACT

The invention relates to a secure system for dispensing customised mixtures to different users, the system being capable of regularly collecting qualified data on the user and on previously mixed dispensed doses to enable the mixture to be adapted to changes in one or more specific parameters of the user. The system comprises a remote server comprising a remote central unit (31) associated with a remote database (32), at least one remote interface (40) for an expert (41), and at least one local automatic dispenser capable of dispensing a customised mixture of products, the dispenser comprising a local data display and data collection interface, and a transmitter/receiver capable of communicating with the remote database.

The invention relates to a secure system for monitoring and extemporaneously dispensing doses of customized pre-determined mixtures of active substances for users, for example, but not exclusively, in the pharmaceuticals, cosmetics and food supplements fields.

Healthcare and cosmetics companies are tending more and more to favor customizing compositions of products according to the final user. This trend is observed for example for the preparation of pharmaceutical treatments specifically tailored to the patient according to their sex, their age, their genetic heritage and specifics of their illness, such as their cancer or the virus strain.

This type of concern also relates to the field of food supplements in order to optimize the effectiveness of these supplements while limiting adverse effects and/or overdoses, and while tailoring the composition of the solution to the effective needs of the patient or the consumer.

There are standard doses or posologies (which, moreover, change depending on the countries). However, these standard doses are not suited to all users. Indeed, it is known that the physiological condition, the genetic heritage and the medical history of the patient need to be taken into account in the posology. Furthermore, these needs are dependent on the progression of the symptoms, the condition, the environment and the behavior of the patient. For all these reasons, customized adjustment of the posology of a treatment according to the effective needs of the user becomes more and more complex and requires the intervention of an expert. The problem is that the more the treatment is customized and the posology tailored to the patient and to their progression, the greater the risk of noncompliance (through oversight or bad interpretation of the posology). Today, there is therefore a restriction to proposing sufficiently customized treatments linked to the risk of breach of the rules of posology that is associated with the risk posed by too complex a customized treatment for the patient to remember or follow.

In order to allow customized preparation of this kind, automatic dispensers comprising at least two cartridges of different components and a mechanical (syringe pump) or pneumatic pump mechanism have already been proposed.

The patent document US6782307 describes a system having a database and Internet access for questioning a customer about their preferences and their personal data and automatically generating a customized formulation that is sent to a production system, which will send the customized preparation to the user, allowing the active substances to be adjusted to meet the effective needs of the patient. However, this system is not found at the user’s house and so dosing is delayed relative to the manufacture thereof and may be irregular, without the system being able to check good compliance with treatment. Moreover, this system does not allow delivery to be made secure at all in the sense that it is not capable of making sure that the user who is identified is indeed the one who needs to receive the customized product or that the user who will take the individualized preparation is indeed the one who has filled in their personal data and preferences. Finally, this system does not allow the posology of the patient to be tailored daily.

The document US20110247718 is also known, which describes a cosmetic product delivery system equipped with a tool for automatically capturing environmental data that tailors the composition of the cosmetic product according to the environmental data. However, this system is not secure because it does not allow overconsumption to be avoided or good compliance with treatment to be checked, or assurance of who is taking the product to be provided. Moreover, it does not take into account the physiological condition of the user, but only weather data.

The document US10022741 describes a dispenser allowing a delivery to be made on demand from multiple cartridges. The user uses the interface to select products that they want and the dispenser provides said products to said user when it detects the presence of a receptacle (any recipient or a hand directly) for receiving the mixture of products.

However, this device is not secure because not only does it allow the delivery of product at the mere request of any user, but it also allows any mixture to be provided. It therefore does not allow the release of a truly tailored mixture to be ensured, or overconsumption to be avoided or good compliance with treatment to be checked, or assurance of who is taking the product to be provided. Moreover, it does not take into account the physiological condition of the user.

The document US20150291411 is also known, which describes a customized delivery system capable of taking customized mixture composition instructions from the user themselves, directly on the machine or via a mobile application on a mobile phone, or of automatically customizing the delivery according to accessible models in a database. However, it does not allow overconsumption to be avoided or good compliance with treatment to be checked, or assurance to be provided that the user who has identified themselves is indeed the one who needs to receive the customized product.

Finally, input of this information is not a condition for obtaining a delivery. Thus, management of a customized treatment based on the immediate condition of the patient cannot be obtained.

Thus, the object of the invention is to propose a system allowing an expert (doctor, nutritionist, etc.) to secure the treatment they prescribe for a patient remotely, while making sure of compliance with the treatment, that is to say while making sure that the behavior of the patient is in keeping with the prescribed treatment (dosing with doses of mixture prescribed in the prescribed posology (quantity, frequency of dosing)), while making sure of who takes the dose, and while allowing the patient to be monitored in order to allow effective updating of the treatment.

One of the problems that the invention proposes solving is therefore that of proposing a system for monitoring and dispensing doses of customized mixtures of products that is capable of ensuring secure compliance with a customized treatment by a patient while improving the regularity of dosing with the doses of mixture and while informing the expert.

In the remainder of the text, a “customized mixture of products” will be understood to mean a customized mixture of active substances in the therapeutical, cosmetic or physiological sense, whether or not the composition of the mixture contains one or more excipients, that is to say elements without therapeutic, cosmetic or physiological effect. An excipient for the purposes of the invention may be, for example, a flavor masker or enhancer, a liquid medium such as water, a fruit juice or a syrup. The aim of the invention is therefore to propose a system for dispensing customized mixtures of products that is secure and controlled, that is to say that the mixture of active substances may not be dispensed to any user, and that the user may not receive any mixture.

The object of the invention is also to improve the customization of the treatment while periodically retrieving, preferably every day, and in a secure manner, data relating to each user and returning them to an expert in charge of creating a customized mixture of products and/or to an algorithm for assisting in the decision as a supplement to the expert.

The object of the invention is also to propose a system for dispensing customized mixtures of products contained in multiple cartridges, to different users without the risk of confusion between said users, in a secure and controlled manner, that is to say that the mixture of active substances may not be dispensed to any user, and that the user may not receive any mixture. Thus, the object of the invention is to allow the dispensing of different customized mixtures to different users, while making sure of compliance with the treatment, of who is taking the dose, and while allowing the patient to be monitored in order to allow effective updating of the treatment.

The object of the invention is also to propose a system for dispensing customized mixtures of products that is capable of regularly collecting verified data concerning the user and past deliveries of mixture in order to allow the mixture to be tailored, by an expert, to the progression of one or more specific parameters of the user.

The object of the invention is also a system for dispensing customized mixtures of products that is capable of ensuring that the information is collected from different users without the risk of confusion between said users.

More precisely, the object of the invention is a secure system for monitoring and dispensing doses of mixtures of active substances, the doses being pre-determined by an expert and customized for users, the system comprising:
a remote server comprising a remote central processing unit associated with a remote database comprising condition information relating to each user, at least one pre-determined composition of a customized mixture of active substances for each user, and posology information comprising authorized periodic dose information for each active substance and/or a timeslot for authorized dosing;
at least one remote interface capable of allowing an expert to create compositions of customized mixtures and capable of communicating with the remote database in transmission mode and in reception mode in order to store therein at least one composition pre-determined by the expert for each user and at least one piece of posology information comprising authorized periodic dose information for each active substance and/or a timeslot for authorized dosing, and capable of communicating with at least one local automatic dispenser (10) of products that is capable of generating a customized mixture of products for at least one user of the dispenser, the or each local automatic dispenser comprising:
active substance tanks;
a local interface for displaying and collecting data provided by the or each user of the dispenser;
a transmitter/receiver capable of communicating with the remote database;
a local central processing unit programmed to selectively actuate each active substance tank according to an instruction to prepare a pre-determined mixture; and
a local memory.

According to particular embodiments:
the system may comprise, moreover, a clock, the local central processing unit and/or the remote central processing unit being connected to the clock and programmed to record in the local memory and/or in the remote database a dispensing timestamp for each dose, and to prevent a new dose or an active substance from being dispensed in the event of an authorized periodic dose being exceeded and/or a dispensing timestamp that is not in accordance with the authorized timeslot for dosing stored in memory;
the remote database may comprise, moreover, temporal compliance data for the dispensing of customized pre-determined mixtures, the local central processing unit or the remote central processing unit being programmed to transmit an alert message for the expert to the remote interface if the user has not ordered the dispensing of a mixture of products for a pre-determined period of time or during the authorized timeslot for dosing, and/or to be able to transmit the compliance data at the request of the expert;
the remote database and/or the local memory may comprise multiple different customized mixtures for a user, and also rules for selecting a composition of a customized mixture according to the condition information relating to the user and/or the timestamps relating to the dosings with doses, the remote central processing unit or the local central processing unit being programmed to compare the condition information relating to the patient and/or the timestamps with the selection rules in order to select the customized mixture tailored to the situation from among multiple mixtures pre-determined by the expert;
the dispensing system may comprise, moreover, an alerting device detectable by a user, the local central processing unit or the remote central processing unit being programmed to actuate the alerting device if the user has not ordered the dispensing of a mixture of products for a pre-determined period of time, or during the authorized timeslot for dosing;
the alerting device may be chosen from among a visual device, an audible device and a haptic device;
the alerting device may be chosen from among a device carried by the dispenser or a device carried by a personal object carried by the user;
the dispenser may comprise, moreover, a presence sensor, the local central processing unit or the remote central processing unit being programmed to actuate the alerting device if the user has not ordered the dispensing of a mixture of products for a pre-determined period of time or during the authorized timeslot for dosing, and if the presence of the user is sensed by the presence sensor;
a personal object of the user and the local automatic dispenser may each comprise a geolocation beacon respectively generating geolocation information relating to the personal object of the user and geolocation information relating to the dispenser, the local central processing unit or the remote central processing unit being programmed to compare the geolocation information relating to the personal object of the user and the geolocation information relating to the dispenser, and to actuate the alerting device if the personal object of the user and the dispenser are spaced by a distance greater than a threshold distance;
the local interface for displaying and collecting data may be carried by the dispenser;
the local central processing unit and/or the remote central processing unit may be programmed, moreover, to display before, during and/or after a dispensing of a customized mixture, on the local interface, questions determined by the expert and to transmit the data relating to the responses to the remote central processing unit and to the remote interface;
the data relating to the responses of the user may be chosen from among the responses themselves, the time of input of the data provided by the user, data relating to the response time for each question and a combination of these;
the remote interface may be capable of allowing an authorized modification of the pre-determined composition of customized mixture of products for the user in order to obtain a suitable composition of customized mixture of products for the user according to the data relating to the responses of the user;
the local central processing unit may be programmed to display questions determined by the expert on the local interface for displaying and collecting data before a dose of customized mixture is dispensed, so as to authorize the delivery of a dose of mixture of products only if the user has previously responded to all of the questions, and to transmit data relating to the responses of the user to the remote central processing unit;
the remote database may comprise identification information relating to each user, and the local dispenser comprises an identification device for identifying a customized security key of a user, the identification device being connected to the local central processing unit, the latter being programmed to check the authenticity of the customized security key, to identify the user associated with this customized security key, and, if the customized security key is authentic, to communicate identification data of the user to the remote server and to receive from the remote server instructions for dispensing a customized pre-determined mixture of products and to order the dispensing of said customized pre-determined mixture of products;
the local central processing unit may be programmed to store, moreover, in a local memory, the identification information, said at least one pre-determined composition of a customized mixture of products for each user, the data provided by the or each user of the dispenser and the condition information relating to each user, and to authorize delivery in the event of disconnection from the communication network, then to transmit the information to the database when the connection is re-established;
the local central processing unit may also be programmed to transmit information for ordering dispensing to the remote database when a customized security key is identified by the dispenser;
the customized security key may be an alphanumeric code and the identification device may be a physical or digital input keypad arranged on the local interface and connected to the central processing unit;
the customized security key may be carried by a personal object of the user and the identification device is capable of identifying the customized security key and of communicating it to the remote central processing unit;
the customized security key may be a visual code and the identification device is a scanner connected to the central processing unit;
the customized security key may be a biometric feature and the identification device is a corresponding biometric sensor;
the customized security key may be a wireless digital signal transmitted by a transmitter carried by the personal object of the user, and the identification device is a wireless receiver connected to the central processing unit for identification;
the wireless digital signal may be a proximity radio signal, the local central processing unit or the remote central processing unit being programmed to identify the wireless digital signal, to evaluate the strength of said signal sensed by the wireless receiver, and to authorize dispensing of a customized mixture only if the strength of said sensed signal is higher than a threshold strength;
the wireless digital signal may be geolocation information relating to the transmitter carried by the personal object of the user, the local automatic dispenser itself comprising a geolocation beacon connected to the local central processing unit and providing geolocation information relating to the dispenser, the local central processing unit or the remote central processing unit being programmed to compare the geolocation information relating to the transmitter carried by the personal object of the user and the geolocation information relating to the dispenser, and to authorize dispensing of a customized mixture only if the transmitter carried by the personal object of the user and the dispenser are spaced by a distance less than a threshold distance; and/or
the personal object of the user may be chosen from among a mobile phone, a smartwatch and an object for collecting the customized preparation.

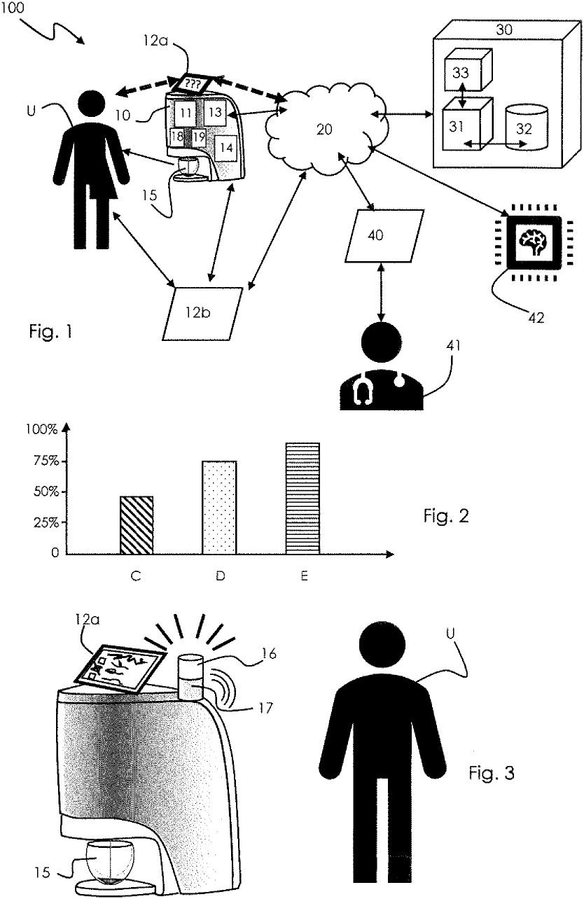

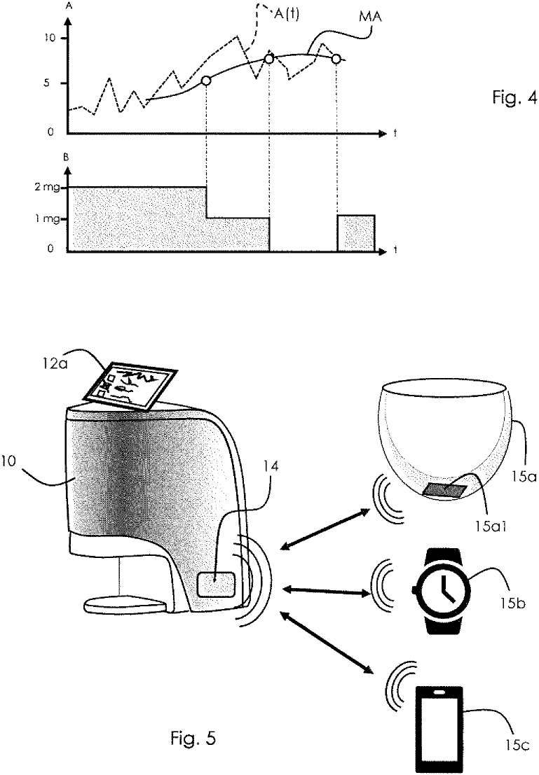

Other features of the invention will be set out in the detailed description below given with reference to the appended figures, which are provided by way of example and in which, respectively:

[FIG. 1] shows a schematic view of a system for dispensing customized pre-determined mixtures of products according to the invention;

[FIG. 2] shows a graph showing the retrieval rate of data according to the local interface used;

[FIG. 3] shows a schematic perspective view of an automatic dispenser equipped with a dosing alerting device;

[FIG. 4] shows two graphs illustrating a process for modifying the customized composition according to a parameter A; and

[FIG. 5] shows a schematic perspective view of an automatic dispenser equipped with a detector for a customized key carried by three embodiments of personal identification objects.

As illustrated in FIG. 1, the invention proposes a system for dispensing customized pre-determined mixtures of products for users. The system 100 comprises one or more automatic product dispensers 10, said dispenser(s) 10 being capable of generating a customized mixture of products for at least one user U of the dispenser. These dispensers are described as local because they are arranged close to the users: home, office, training room, etc.

Each product dispenser 10 is capable of communicating (in transmission mode and in reception mode, as indicated by the double-headed arrows in FIG. 1) with a remote server 30, comprising a remote central processing unit 31 associated with a remote database 32, via a telecommunication network 20, such as the Internet. Said database comprises at least one pre-determined composition of a customized mixture of products for each user U, authorized dosing timeslot information and/or posology information comprising authorized periodic dose information for each active substance and/or a timeslot for authorized dosing and, advantageously, identification information relating to each user U.

The remote database 32 also comprises condition information relating to each user, obtained from the user themselves following a series of questions posed to them through a local interface 12 in the form of a screen 12a carried by the product dispenser.

Optionally, the remote database 32 may also comprise information obtained by one or more sensors carried by the users (smartwatch, sports activity sensor, heart rate sensor, for example).

The condition information may vary according to the user and, in particular, the reasons for which they take the customized mixture. For example, if they take a mixture that aims to improve their sleep, the condition information may comprise in particular monitoring of the sleep phases or a self-evaluation of the tiredness of the user. If they take a mixture that aims to alleviate the operation of their liver, the condition information may in particular comprise monitoring of the composition of their meals and their alcohol consumption. If they take a mixture that aims to restore their intestinal balance (their microbiota), the condition information may in particular comprise monitoring of flatulence symptoms or constipation symptoms and their consumption of fiber-rich products. In another field, the condition information may comprise for example questions relating to the side-effects of a treatment (for example during chemotherapy) in order to adjust the active substances or to warn the expert of a problem and of the need for a second examination.

More generally, the condition information relates to at least one parameter such as a physiological, physical or psychic parameter of each user.

The system according to the invention also comprises at least one local interface 40 for an expert 41, capable of allowing the expert 41 to create compositions of customized mixtures, and capable of communicating in transmission mode and in reception mode with the remote database 32 in order to store therein at least one pre-determined composition created by the expert for each user and at least one piece of posology information comprising authorized periodic dose information for each active substance and/or a timeslot for authorized dosing, and capable of communicating with at least one local automatic dispenser 10, directly or indirectly (via the network 20 and/or via the remote server 30).

The system according to the invention therefore secures the dispensing of the mixture because it is a human expert 41 who is in charge of creating one or more customized mixtures of products. In one particular embodiment, the system according to the invention may also comprise a decision-assistance algorithm 42 programmed to make a proposal to the human expert 41 via the remote interface 40, the expert being able either to confirm the proposal or to modify it via the remote interface 40. This algorithm is advantageously based on artificial intelligence that automatically creates a proposal for modifying the customized mixture on the basis of data received from the local automatic dispenser 10, as described later on. The remote interface 40 is capable of communicating in transmission mode and in reception mode, through the communication network 20, with the remote database 32 in order to store therein at least one composition determined by the expert for each user, and capable of communicating with at least one local automatic dispenser 10 of products so that the latter is able to generate a customized mixture of products for at least one user of the dispenser.

If the system comprises a decision-assistance algorithm 42, the latter may be programmed either directly in the server 31 or in an independent server capable of communicating with the remote database 32 in transmission mode and in reception mode through the communication network 20.

The or each local automatic dispenser comprises a frame fitted with N locations (not shown) for tanks of products (active substances and, possibly, excipients), for example cartridges of products that are liquid or in the form of pills, powder or paste, N being an integer greater than or equal to 2. The dispenser 10 according to the invention also comprises a motor (not shown) capable of generating sufficient force to selectively actuate the cartridges and generate the delivery.

Of course, the concern for the invention is to provide for a large number of tank locations in order to be able to use more different products and allow the delivery of a large number of combinations of mixtures. Thus, N is advantageously chosen to be between 3 and 8. The number of tank locations is limited by the size of the machine, the power of the motor and the size of the cartridges, which need to remain sensible, especially if the dispenser is intended for domestic use.

Advantageously, each active substance tank (or cartridge) comprises an identification means, such as an electronic identification chip, and the dispenser comprises a means for reading each identification means of the cartridges, such as a sensor capable of reading identification chips. The dispenser 10 (or the remote server 30) is thus able to identify each tank and to know its location in the dispenser.

Moreover, the dispenser 10 according to the invention comprises a central processing unit 11 programmed to selectively actuate each tank location according to an instruction to prepare a mixture in a determined manner.

When the tanks are fitted with identification chips for identifying the product, the local central processing unit 11 is programmed to selectively actuate each active substance tank identified by its chip according to the sensed identification information and an instruction to prepare a pre-determined mixture.

Courtesy of these identification means for identifying the cartridges, and therefore the products contained in the cartridges, the system according to the invention allows possible interactions between products, in particular harmful interactions, to be managed by avoiding mixtures of products that are incompatible with one another. For example, certain nutriments may have opposite effects (exciting or relaxing) and it is desirable to be able to prevent these nutriments from being delivered concomitantly, which the invention allows. Thus, the local central processing unit 11 and/or the remote central processing unit 31 is/are programmed to compare the prescription with a list of interactions that is stored in the local memory 18 and/or the remote memory 32, and to prohibit one or more active substances from being dispensed in the event of incompatibility. Advantageously, in this case, the system warns the expert 41.

According to the invention, the or each local automatic dispenser also comprises a local interface 12, for displaying and collecting data provided by the or each user of the dispenser, and comprises a transceiver 13 capable of communicating with the remote database 32 via the telecommunication network 20.

As described hereinabove, the local interface 12 is advantageously preferably a screen 12a carried directly by the dispenser, that is to say forming an integral part of the dispenser, without the possibility of removing it from the dispenser.

In combination, and as security, the system may comprise, moreover, a local assistance interface (in case of a problem encountered by the screen 12a of the dispenser), such as the screen 12b of the mobile phone of the user or of their personal computer, subject to its being programmed in order to communicate (in transmission mode and in reception mode) with the dispenser 10 and/or the remote server 30 in order to display and collect information. If a problem is encountered by the screen 12a of the dispenser concomitantly with a loss of Internet connection by the dispenser 10, then the screen 12b will have to be programmed in order to necessarily communicate (in transmission mode and in reception mode) with the dispenser 10.

Alternatively, the local central processing unit 11 and the local transceiver 13 may be programmed to communicate with the mobile phone of the user, which is itself programmed to share its connection and allow the local central processing unit 11 to communicate with the remote central processing unit 31 via the mobile phone of the user. In this case, the local interface remains the screen 12a carried directly by the dispenser.

FIG. 2 illustrates the effect obtained courtesy of a local interface 12 in the form of a screen 12a carried directly on the dispenser. Indeed, the first hatched bar C corresponds to the retrieval rate for condition information relating to the patient with a local interface constituted by the screen 12b of a mobile phone. This rate is lower than 50%, which is very little. This means either that the users return no information (poor quantity of information) or that the input information is not relevant (poor quality of the information). By contrast, the use of a screen 12a carried directly by the dispenser (dotted bar D) ensures a retrieval rate for condition information relating to the patient that is higher than 75%. This means that the users are more inclined to respond, and that they respond better.

This rate even goes beyond 90% when the dispensing of the customized mixture is based on the retrieval of condition information relating to the patient. Indeed, in this case, the user is obliged to respond to the questions in order to obtain the mixture, and the responses need to be of good quality. Thus, the user is more inclined to respond to the questions posed on their dispenser than on their mobile phone. The number of responses and the quality of the responses obtained have a direct and significant effect on compliance with the treatment by the user, on the quality of the mixture composed by the expert and on the responsiveness of the modification of the mixture by the expert.

Thus, the fact that the screen is carried by the dispenser makes it possible to make sure that the user who responds to the questions is close to the dispenser and that it is indeed said user who is taking the dispensed mixture.

This allows a multi-user implementation of the dispenser, ensuring that customized mixtures of products contained in multiple cartridges are dispensed to different users without the risk of confusion between said users, in a secure and controlled manner. Thus, the customized mixture of active substances may not be dispensed to any user, and the user may not receive any mixture, while making sure of compliance with the treatment, of who takes the dose, and while allowing the patient to be monitored in order to allow effective updating of the treatment.

To ensure the user’s safety, the system according to the invention allows any overconsumption or any bad dosing with one or more active substances to be avoided.

To this end, the invention provides for the remote database 32 to be able to comprise, moreover, temporal compliance data for the dispensing of customized pre-determined mixtures. To this end, the remote server 30 and/or the local dispenser 10 is/are also fitted with a clock 33 or 19, respectively. The local central processing unit 11 and/or the remote central processing unit 31 is/are connected to the clock 19, 33 and is/are programmed to record in the local memory 18 and/or in the remote database 32 a time and a date of dispensing for each dose.

Thus, given that the system 100 is fitted with a clock 19-33, the local central processing unit 11 and/or the remote central processing unit 31 is/are also programmed to record a dispensing timestamp for each dose (the time and the date of the dispensing of each dose) and to prevent a new dose or an active substance of the dose from being dispensed in the event of a periodic dose (for example daily, weekly, monthly, or other) authorized by the expert 41 being exceeded, and/or a dispensing timestamp that is not in accordance with the previously authorized timeslot for dosing defined by the expert and stored in the remote database 32 and/or in the local memory 18.

For example, the dispensing of melatonin (active substance) may be blocked if the time of dosing is in the morning. Also, for example the dispensing of a stimulant (such as turmeric, which stimulates the secretion of serotonin and dopamine) may be blocked if the time of dosing is in the evening.

In the pharmaceuticals field, it is known for example that, for an active substance such as paracetamol, an overdose may cause serious lesions of the liver. The posology in terms of dose per dosing and frequency of dosing is therefore dependent on the weight and age of the patient. Now, users often make mistakes over dosage or forget the time of the last dosing. A device according to the invention will allow the user not to make mistakes over doses and will automatically block re-dosing with this anti-inflammatory when attempted dosings are too close, which will allow the harmful side-effects linked to breach of the strict posology to be avoided.

In the cosmetics field, for the application of topical agents, for example calcipotriol in cream form (used in the treatment of plaque psoriasis affecting up to 40% of the body surface area), systemic effects (hypercalcaemia and hypercalciuria) have been reported: the risk of presenting systemic effects increases if the total dose increases, but the therapeutic effect increases as the dose received locally increases. It is therefore necessary, for an expert, to evaluate the right dose of cream and of active substance to administer to the user according to the severity of the psoriasis, the surface area to be covered by the treatment and the risk faced by the user of developing systemic effects. With the system according to the invention, the dosage of the cream will be able to be systematically implemented in line with the recommendations of the expert. If the user were to present areas of psoriasis with different levels of severity, a system according to the invention will allow multiple deliveries to be proposed and the dosage of the active substance of the total quantity of cream to be tailored independently. The system according to the invention will even be able, following the questions posed to the user, to progressively adjust the dosage of the active substance to match the reduction according to the progression of the psoriasis reported by the user according to each area treated.

Generally, other posological rules will be able to be collected by the system according to the invention and used to automatically adjust the dosage according to clearly identified cases. For example, it will be possible to define minimum dosing intervals. In the case of an active substance such as paracetamol, the expert will be able to define, instead of a given and fixed dosing slot, a minimum dosing interval (for example at least equal to 4 h). Thus, the system according to the invention will allow the customized mixture to be released at the most suitable moment according to the symptoms felt by the user (fever in the example of paracetamol) and will block any delivery while the minimum dosing interval is not observed.

Advantageously, an alert message is displayed on the local interface 12a in order to warn the user that the periodic dose is exceeded. This may be useful, for example, for users suffering from memory impairment.

It may also be useful to program the system to allow an incident procedure if, for example, the user spills the dose before swallowing it. Indeed, in this case, the periodic dosing dose is not exceeded, but the periodic dispensing dose may be so. In one embodiment, the local central processing unit is programmed to display an incident declaration request on the local interface 12a. Once this request has been selected by the user, the local central processing unit 11 sends an incident alert to the remote server, which responds by returning a message warning the user of the exceeding and notifying them of the cause, for confirmation on the local interface 12a. Once the declaration of an incident and declaration of responsibility being taken by the user has been confirmed and returned to the remote server 30, the latter authorizes another dispensing of a dose. Advantageously, the authorization of another dispensing may take into account the time between the moment at which the normal dose was first dispensed and the moment at which the declaration of an incident appeared. Thus, if the declaration of an incident occurs after a pre-determined period of time after normal release, re-release is denied as a security measure.

In combination, the local central processing unit 11 and/or the remote central processing unit 31 are also programmed to transmit compliance data for the dosing with the mixture to the expert 41 via their interface 40 and, possibly, to the decision-assistance algorithm 42, and to transmit an alert message if the user has not ordered the dispensing of a mixture of products for a pre-determined period of time or during the authorized timeslot for dosing. Alternatively or in combination, the remote central processing unit 31 may also be programmed to transmit an alert message to a referenced and authorized third party (for example a care assistant or a close relative) if the user has not ordered the dispensing of a mixture of products for a pre-determined period of time or during the authorized timeslot for dosing, for example in the case of a prescription for an elderly person.

Alternatively or in combination, the posology information recorded by the expert 41 via their remote interface 40 in the remote database 32 may comprise minimum periodic dose information for at least some of the active substances. In this way, when the user has not taken a prescribed dose, the system is able to increase the quantity of one or more active substance(s) in the next dose. For example, when the doctor prescribes a dose of magnesium in the morning and in the evening in order to distribute the dosings and the user forgets to take their dose in the morning, the system according to the invention is able to automatically release a double quantity of magnesium in the evening dose.

Alternatively or in combination, in an advantageous embodiment of the invention, shown in FIG. 3, the system according to the invention comprises an alerting device 16 detectable by a user U.

In the embodiment shown in FIG. 3, the alerting device 16 is carried by the dispenser. Moreover, the local dispenser 10 is fitted with a clock 19 and the local central processing unit 11 is programmed to actuate the alerting device 16 if the user has not ordered the dispensing of a mixture of products for a pre-determined period of time or during the authorized timeslot for dosing.

In this way, when the user passes by close to the dispenser, they think of ordering their customized mixture and of responding to the questions that are posed.

The alerting device may be chosen from among a visual device, an audible device and a haptic device or a combination of these.

Advantageously, the alerting device may be active for as long as the person has not returned their data and taken their food supplements.

In an advantageous embodiment, the dispenser 10 comprises, moreover, a presence sensor 17, the local central processing unit 11 being programmed to actuate the alerting device 16 if the user has not ordered the dispensing of a mixture of products for a pre-determined period of time or during the authorized timeslot for dosing and if the presence of the user U is sensed by the presence sensor 17.

Alternatively or in combination, the system is able to evaluate the distance between the user and the dispenser and to actuate the alerting device if the user moves away without having taken the prescribed dose.

To this end, a personal object 15a, 15b, 15c (see FIG. 5) of the user and the local automatic dispenser each comprise a geolocation beacon generating geolocation information relating to the personal object 15a, 15b, 15c of the user and relating to the dispenser 10. The local central processing unit 11 or the remote central processing unit 31 is then programmed to compare the geolocation information relating to the personal object 15a, 15b, 15c of the user and the geolocation information relating to the dispenser 10, and to actuate the alerting device 16 if the personal object 15a, 15b, 15c of the user (and therefore the user themselves) and the dispenser 10 are spaced by a distance greater than a threshold distance. For example, this allows a user to be reminded that they have forgotten to take their customized mixture of products before definitively leaving their home.

Alternatively or in combination, the alerting device may be comprised in an object carried by the user, such as for example their mobile phone. In this case, the telephone rings, vibrates or flashes when the user has not ordered the dispensing of a mixture of products for a pre-determined period of time or during the authorized timeslot for dosing. In this case too, the alerting device may be triggered if the telephone, and therefore its user, moves away from the dispenser by comparing the geopositioning data relating to the telephone and relating to the dispenser. The invention also allows effective and certain tailoring of the mixture in the course of the treatment by allowing quality information to be collected, at regular intervals.

To this end, the local central processing unit 11 is programmed to display, before, during and/or after a dispensing of a customized mixture, on the local interface 12a, questions pre-determined by the expert 40 and to transmit the responses to the remote central processing unit 31.

Preferably, the local central processing unit 11 may be programmed to authorize delivery of a mixture of products only if the user has previously responded to all of the questions. To this end, the local central processing unit 11 is programmed to display questions determined by the expert 41 on the local interface 12a for displaying and collecting data before a dose of customized mixture is dispensed, so as to authorize delivery of a dose of mixture of products only if the user has previously responded to all of the questions, and to transmit data relating to the responses of the user to the remote central processing unit 31.

The data relating to the responses of the user may be the responses themselves, but also advantageously, courtesy of the clock 19 (or 33), data relating to the response time for each question, or even the time of response in order to allow an analysis of the rhythm of life and possible tailoring of the mixture by the expert. It is indeed possible to characterize physical, physiological or mental conditions according to the time taken by the user to respond to such and such a question. The data relating to the responses of the user may also be comparisons of response times for one and the same question at different intervals in order to deduce therefrom a progression of the response time over time according to the treatment.

The system according to the invention is thus suitable for allowing modification of the pre-determined composition of customized mixture of products for the user in order to obtain a tailored composition of customized mixture of products for the user according to the data relating to the responses of the user.

The remote central processing unit 31 may be programmed to transmit to the expert 41, via their remote interface 40, and, possibly, to the decision-assistance algorithm 42, the data relating to the responses of the user so that the expert is able to decide whether or not to modify the mixture, in what proportions and in what posology (quantity and frequency or time of dosing), possibly using proposals made by the decision-assistance algorithm.

These modifications authorized by the expert are stored in the local memory 18 and/or in the remote database 32, and the local central processing unit 11 is programmed to selectively actuate each active substance tank according to an instruction to prepare a pre-determined mixture including these authorized modifications according to the data relating to the questions that are provided by the user.

In an example of a customized food supplement, the user has a dispenser 10 at their home, which has been programmed remotely by their expert nutritionist 41. In a standard mode of operation, the user has two deliveries per day, one in the morning and one in the evening in order to support them during the day and at night.

In order to tailor the program of the user, the nutritionist needs to know whether the user has restorative sleep and whether the user is stressed. When delivering their food supplement in the morning, the machine asks the user, via its touch interface 12, to grade the quality of their sleep the previous night from 1 to 10. Equally, when delivering their food supplement in the evening, the interface asks the user to grade their level of stress during the day from 1 to 10.

The data relating to the user are stored in the remote database 31, and at the end of their first month of treatment, when visiting their expert nutritionist, the user allows the latter to access this health information in order to adjust their recommendation via a web or mobile interface. By observing the progression of the quality of sleep and of the quantity of stress felt by the user, the healthcare professional will be able to adjust the customized program by increasing or reducing the quantity of certain food supplements. These new recommendations are stored in the remote database 31 so that they may be requested by the automatic dispenser.

For example, if stress has not reduced sufficiently, they will be able to increase the doses of certain plants, such as rhodiola, which is known for its de-stressing properties.

Conversely, if the quality of sleep has returned to normal, it will be able to reduce or even stop the use of certain food supplements such as melatonin.

Advantageously, the expert 41 is able to define multiple compositions of mixtures customized for a user that are stored in the remote database 32 and/or in the local memory 18, and rules for selecting a composition of a customized mixture according to the data relating to the condition information from the user (for example the user’s responses to the questions posed) and/or the timestamps relating to the dosings with doses.

These rules are stored in the remote database 32 and/or in the local memory 18 by the user via the remote interface 40, and the remote central processing unit 31 or the local central processing unit 11 is programmed to compare the condition information relating to the patient and/or the timestamps with these rules in order to select, according to the rules defined by the expert, the customized mixture tailored to the situation from among multiple mixtures pre-determined by the expert.

FIG. 4 shows an example of rules for modifying a composition of a customized mixture (parameter B) according to a parameter A, for example by adjusting the dose of certain food supplements according to the results of the evaluations.

For example, in FIG. 4, the grades relating to sleep (parameter A) are reported in the graph at the top and form the dashed curve A(t).

The local central processing unit 11 or the remote central processing unit 31 then calculates the mean of the grades over seven rolling days, which generates the solid curve MA.

If the mean sleep quality MA over the last 7 days is higher than 5, the evening dose of melatonin (parameter B) will be 1 mg. If the mean sleep quality MA is lower than 5, the evening dose of melatonin (parameter B) will be 2 mg; if the mean sleep quality MA is higher than 8, the evening dose of melatonin (parameter B) will be 0 mg.

Another process for modifying the possible customized composition might consist, for example, in counting the number of events over a given period. By asking a user whether they have indeed drunk 2 L of water the day before, it is confirmed for example that they have drunk enough water to eliminate the toxins from their body. Over a period of 3 consecutive days on which the water consumption has not been reached, it will be possible to adjust the intake of vitamins and minerals so as not to overload the kidneys.

For that, it is necessary to have regular data, without which the recommendation risks being distorted. Indeed, if the user fills in their evaluation only when they have slept badly, this will tend to increase the doses of melatonin needlessly, in light of the fact that it is not medically recommended to use melatonin in a very high dose for too long.

It is therefore essential to collect the information regularly. Using the delivery machine according to the invention with an interrogation interface 12a greatly improves the acquisition of information and the quality of the treatment. Indeed, when the user is waiting for their customized delivery, they are more inclined to respond to these evaluations. As described hereinabove, it has been noted that the use of the interface 12a of the machine rather than a web or mobile interface 12b very clearly improves the retrieval of daily information.

Thus, the invention allows the customization of recommendations by a professional to be improved, possibly using a decision-assistance algorithm. In the case of elements having a risk of overdose, these daily evaluations may become essential in order to avoid harmful overdoses for the user.

The information collected daily may be declaratory (evaluation, grading), extrapolated (time of dosing, responsiveness in relation to the interface, etc.) or sensed by sensors integrated in the machine (pulse test, image of the face, glucose or conductivity test).

In the case of a customized cosmetics preparation, one example might consist in asking the user whether they feel tightness in the skin (yes or no). At the end of one month of use, the expert (the healthcare professional) will be able to evaluate, possibly using an algorithm, whether the tightness is occasional, regular or localized to certain periods (for example when it is cold or the day after sports activities) and will be able to adjust the customized program according to the analysis of this daily information obtained over a sufficiently long period.

In medical use, it will be possible for example to collect the psychological perception of a user in order to progressively tailor the prescription of drugs, for example anxiolytics.

In addition to making dosing with doses secure, the invention may, moreover, allow the dispensing to be made secure by making sure that the user who has identified themselves is indeed the one who needs to receive the customized mixture of product.

The or each local automatic dispenser 10 also comprises an identification device 14 for identifying a local customized security key 15 of a user, and the remote database 32 comprises, moreover, identification information relating to each user U.

Local customized security key is understood, according to the invention, to mean an identification means close to the dispenser. Multiple embodiments are illustrated in FIG. 5 and will be described later on.

When the identification device 14 is connected to the local central processing unit 11, the latter is programmed to check the authenticity of the local customized security key, to identify the user associated with this local customized security key and, if the local customized security key is authentic, to communicate identification data relating to the user to the remote server 30 and receive from the remote server 30 instructions to dispense a customized determined mixture of products and to order the dispensing of said customized determined mixture of products by the dispenser 10.

In order to be authentic, the security key needs to be unique to a user/password pair.

The local customized security key of the present invention, which is input by the user, directly or indirectly, in order to identify themselves and in order that they may receive a mixture of products that is pre-determined by an expert, must not, of course, be confused with order data that allow any user to compose a mixture themselves and to order the dispensing of the mixture that they have composed, without any identification of said user.

Indeed, according to the invention, the user does not have the choice of composing the mixture(s) of active substances pre-programmed by the expert. This composition is determined or authorized by the expert, prior to dispensing, so that the dispensing is secure and easy for any user, including those having no knowledge relating to the mixture of active substances.

On the other hand, in an optional embodiment, the dispenser and the system according to the invention may be configured so that the user is able to add or remove any excipient of their choice dispensed by the dispenser, such as, for example, a flavor masker or enhancer, a liquid medium such as water, a fruit juice or a syrup, etc. In the case of cosmetics, this may be for example a pigment allowing the preparation to be colored according to the skin complexion of the user U.

The system according to the invention may also allow the expert to program multiple mixtures predefined by the user (different active substances and/or different dosages). Indeed, it may be desirable to tailor dosing according to certain circumstances. In this case, the expert programs multiple predefined mixtures that the user needs to use according to the circumstances. For example, if a particular event occurs (chronic symptom, fever, etc., and so on), the user will be able to choose from among the predefined mixtures. It will be advantageous to program a question into the interface in order to guide the user toward the most advantageous predefined mixture. However, if the user is able to choose the mixture according to the circumstances (and possibly the excipients), they do not, even so, have the choice of composition of active substances of the predefined mixture that is chosen. It is the expert who has pre-determined this composition via their interface 40.

Also according to the invention, the mixture is determined a first time a priori, at the start of treatment, by the expert 41 via their remote interface 40, and said determination may be modified and tailored a posteriori, in the course of the treatment, by the expert 41, possibly using an algorithm 42 programmed for example in the remote server 30.

For this tailoring to be effective, it is necessary for the system to retrieve data from the user, either directly or indirectly by way of sensors carried by the user.

To this end, the invention proposes making delivery of the mixture secure by making sure that the user who has activated the dispenser is indeed the one who needs to receive the mixture, and making the collection of data secure in order to make sure that said data are qualified, that is to say that they are of good quality and precisely reflect the physiological, physical and/or mental situation of the user U.

In order to make delivery secure, the or each local automatic dispenser 10 also comprises an identification device 14 for identifying a local customized security key 15 of a user. Additionally, the local central processing unit is also programmed to transmit information for ordering dispensing to the remote database when the customized security key is identified by the dispenser. Alternatively or in combination, the central processing unit may locally store, in a local memory 18, the identification parameters, the delivery information, the delivery orders and the condition information in order to be able to ensure delivery in the event of temporary disconnection from the communication network 20, and to later restore the information to the database 32 when the connection is re-established.

In a first embodiment, the customized security key is an alphanumeric code and the identification device 14 is a physical or digital input keypad arranged on the local interface 12a and connected to the central processing unit 11.

Thus, once the user has been identified by the remote server, the system sends to the mobile phone or the computer of the user the customized security key in the form of an alphanumeric code to be input on the local interface 12a. Thus, the system according to the invention makes sure that the person who has input the identification information is also the person for whom the customized security key is intended.

Preferred embodiments, shown in FIG. 5, allow faster security without active intervention from the user. These embodiments use an object personal to each user, the customized security key being carried by this personal object. Additionally, the identification device is capable of automatically identifying the customized security key and of communicating it to the remote central processing unit 31.

FIG. 5 shows three embodiments in which the personal object of the user is, respectively, a recipient for collecting the customized preparation 15a, a smartwatch 15b and a mobile phone 15c.

The customized security key may be a visual code 15a1 carried by the recipient 15a or displayed on the screen of the smartwatch 15b or of the phone 15c. It may be, for example, a barcode or a QR code. Additionally, the identification device 14 is then a scanner capable of sensing the visual code and transmitting it to the remote central processing unit 30 and/or to the local unit 11 if the key is stored in the dispenser 10.

Alternatively, the customized security key may be a wireless digital signal (for example a near field communication, or NFC, signal or even a Bluetooth signal, a geolocation signal, etc.) transmitted by a transmitter carried by the personal object of the user. For example, it may be an RFID chip glued to the recipient or integrated in the recipient 15a. Additionally, the identification device 14 is a wireless receiver capable of communicating with the remote central processing unit 31 or the local central processing unit 11 for identification.

Preferably, in the embodiment in which the wireless digital signal is a proximity radio signal (NFC or Bluetooth, for example), the local 11 or remote 31 central processing unit is programmed to identify the wireless digital signal, but also to compare the strength of said signal sensed by the wireless receiver 13 with a threshold strength. Moreover, the local central processing unit 11 or the remote central processing unit 31 is programmed to authorize dispensing of a customized mixture only if the strength of said signal sensed by the identification device 14 is higher than a threshold strength.

If it is the local central processing unit 11 that compares the strength of the sensed signal with a threshold strength, said unit is programmed to communicate with the remote central processing unit 30 and to order release of the mixture only if the strength of said sensed signal is higher than a threshold strength.

If it is the remote central processing unit that compares the strength of the sensed signal with a threshold strength, said unit is programmed to send the authorization for release of the mixture to the local central processing unit 11 only if the strength of said sensed signal is higher than a threshold strength.

Alternatively or in combination, the wireless digital signal may be geolocation information relating to the transmitter carried by the personal object of the user. In this case, the local automatic dispenser itself comprises a geolocation beacon connected to the local central processing unit 11 or any other geolocation means (IP address, and so on) for providing geolocation information relating to the dispenser 10.

The local 11 or remote 31 central processing unit is then programmed to compare the geolocation information relating to the transmitter carried by the personal object 15a, 15b, 15c of the user and the geolocation information of the dispenser 10, and to authorize dispensing of a customized mixture only if the transmitter carried by the personal object 15a, 15b, 15c of the user and the dispenser are spaced by a distance less than a threshold distance.

If it is the local central processing unit 11 that compares the geolocation positions, said unit is programmed to communicate with the remote central processing unit 30 and to order release of the mixture only if the transmitter carried by the personal object 15a, 15b, 15c of the user and the dispenser are spaced by a distance less than a threshold distance.

If it is the remote central processing unit that compares the geolocation positions, said unit is programmed to send the authorization for release of the mixture to the local central processing unit 11 only if the transmitter carried by the personal object 15a, 15b, 15c of the user and the dispenser are spaced by a distance less than a threshold distance.

Alternatively, the customized security key may be a biometric feature and the identification device 14 is then a corresponding biometric sensor. For example, this biometric feature may be a fingerprint, a face, an iris, etc.

Alternatively, the customized security key may be a physical security key, such as a key, a chip card, a magnetic badge. In this case, the local central processing unit is programmed to transmit information for ordering dispensing to the remote database when the customized security key is input into the dispenser.

Alternatively, the customized security key may be a combination of the previous security keys, which allows security to be increased.

Thus, the system according to the invention allows an expert (doctor, nutritionist, etc.) to make the treatment that they prescribe for a patient remotely more secure by allowing them to effectively monitor compliance with the treatment, by making sure of who takes the dose and by allowing the patient to be monitored in order to allow customized updating of the treatment.

The system according to the invention also allows the regularity of dosing with the doses of mixture to be improved by informing the expert and by reminding the user to take their doses.

The system according to the invention also allows the customization of the treatment to be improved by periodically retrieving good-quality data relating to each user, and by restoring said data to an expert so that they may possibly tailor the prescription to the actual condition of the patient, which allows complex treatments to be administered without risking confusion for the user.

The invention also allows proposal of a system for dispensing customized mixtures to different users that avoids the risks of confusion among said users.

1. A secure system (100) for monitoring and dispensing doses of mixtures of active substances, the doses being pre-determined by an expert and customized for users, characterized in that it comprises:
a remote server (30) comprising a remote central processing unit (31) associated with a remote database (32) comprising condition information relating to each user, at least one pre-determined composition of a customized mixture of active substances for each user, and posology information comprising authorized periodic dose information for each active substance and/or a timeslot for authorized dosing;
at least one remote interface (40) capable of allowing an expert (41) to create compositions of customized mixtures and capable of communicating with the remote database (32) in transmission mode and in reception mode in order to store therein at least one composition pre-determined by the expert for each user and at least one piece of posology information comprising authorized periodic dose information for each active substance and/or a timeslot for authorized dosing, and capable of communicating with at least one local automatic dispenser (10) of products that is capable of generating a customized mixture of products for at least one user of the dispenser,
the or each local automatic dispenser (10) comprising:
active substance tanks;
a local interface (12, 12a, 12b) for displaying and collecting data provided by the or each user of the dispenser;
a transmitter/receiver (13) capable of communicating with the remote database (32);
a local central processing unit (11) programmed to selectively actuate each active substance tank according to an instruction to prepare a pre-determined mixture; and
a local memory (18).

2. The dispensing system as claimed in claim 1, further comprising a clock (19, 33), the local central processing unit (11) and/or the remote central processing unit (31) being connected to the clock (19, 33) and programmed to record in the local memory (18) and/or in the remote database (32) a dispensing timestamp for each dose, and to prevent a new dose or an active substance from being dispensed in the event of an authorized periodic dose being exceeded and/or a dispensing timestamp that is not in accordance with the authorized timeslot for dosing stored in memory.

3. The dispensing system as claimed in claim 2, wherein the remote database further comprises temporal compliance data for the dispensing of customized pre-determined mixtures, the local central processing unit (11) or the remote central processing unit (31) being programmed to transmit an alert message for the expert (41) to the remote interface (40) if the user has not ordered the dispensing of a mixture of products for a pre-determined period of time or during the authorized timeslot for dosing, and/or to be able to transmit the compliance data at the request of the expert (41).

4. The dispensing system as claimed in claim 2 wherein the remote database (32) and/or the local memory (18) comprises, multiple different customized mixtures for a user, and also rules for selecting a composition of a customized mixture according to the condition information relating to the user and/or the timestamps relating to the dosings with doses, the remote central processing unit (31) or the local central processing unit (11) being programmed to compare the condition information relating to the patient and/or the timestamps with the selection rules in order to select the customized mixture tailored to the situation from among multiple mixtures pre-determined by the expert.

5. The dispensing system as claimed in claim 2 further comprising an alerting device (16) detectable by a user, the local central, processing unit (11) or the remote central processing unit (31) being programmed to actuate the alerting device if the user has not ordered the dispensing of a mixture of products for a pre-determined period of time, or during the authorized timeslot for dosing.

6. The dispensing system as claimed in claim 5, wherein the alerting device is chosen from among a visual device, an audible device and a haptic device.

7. The dispensing system as claimed in claim 5, wherein the alerting device is chosen from among a device carried by the dispenser or a device carried by a personal object (15a, 15b, 15c) carried by the user.

8. The dispensing system as claimed in claim 5, wherein the dispenser further comprises a presence sensor, the local central processing unit (11) or the remote central processing unit (31) being programmed to actuate the alerting device if the user has not ordered the dispensing of a mixture of products for a pre-determined period of time or during the authorized timeslot for dosing, and if the presence of the user is sensed by the presence sensor.

9. The dispensing system as claimed in claim 5, wherein a personal object (15a, 15b, 15c) of the user and the local automatic dispenser each comprise a geolocation beacon respectively generating geolocation information relating to the personal object (15a, 15b, 15c) of the user and geolocation information relating to the dispenser (10), the local central processing unit (11) or the remote central processing unit (31) being programmed to compare the geolocation information relating to the personal object (15a, 15b, 15c) of the user and the geolocation information relating to the dispenser (10), and to actuate the alerting device (16) if the personal object (15a, 15b, 15c) of the user and the dispenser (10) are spaced by a distance greater than a threshold distance.

10. The dispensing system as claimed in claim 1, wherein the local interface (12a) for displaying and collecting data is carried by the dispenser.

11. The dispensing system as claimed in claim 1 wherein the local central processing unit (11) or the remote central processing unit (31) is programmed, moreover, to display before, during and/or after a dispensing of a customized mixture, on the local interface (12a), questions determined by the expert (41) and to transmit the data relating to the responses to the remote central processing unit (31) and to the remote interface (40).

12. The dispensing system as claimed in claim 11, wherein the data relating to the responses of the user are chosen from among the responses themselves, the time of input of the data provided by the user, data relating to the response time for each question and a combination of these.

13. The dispensing system as claimed in claim 11 , wherein the remote interface (40) is capable of allowing an authorized modification of the pre-determined composition of customized mixture of products for the user in order to obtain a suitable composition of customized mixture of products for the user according to the data relating to the responses of the user.

14. The dispensing system as claimed in claim 11, wherein the local central processing unit is programmed to display questions determined by the expert on the local interface for displaying and collecting data before a dose of customized mixture is dispensed, so as to authorize delivery of a dose of mixture of products only if the user has previously responded to all of the questions, and to transmit data relating to the responses of the user to the remote central processing unit.

15. The dispensing system as claimed in claim 1, wherein the remote database (32) comprises identification information relating to each user (U), and the local dispenser (10) comprises an identification device (14) for identifying a customized security key of a user, the identification device (14) being connected to the local central processing unit (11), the latter being programmed to check the authenticity of the customized security key, to identify the user associated with this customized security key, and, if the customized security key is authentic, to communicate identification data of the user to the remote server (30) and to receive from the remote server instructions for dispensing a customized pre-determined mixture of products and to order the dispensing of said customized pre-determined mixture of products.

16. The dispensing system as claimed in claim 15, wherein the local central processing unit (11) is programmed to store, moreover, in a local memory (18), the identification information, said at least one pre-determined composition of a customized mixture of products for each user, the data provided by the or each user of the dispenser and the condition information relating to each user, and to authorize delivery in the event of disconnection from the communication network (20), then to transmit the information to the database (32) when the connection is re-established.

17. The dispensing system as claimed in claim 15 , wherein the local central processing unit is also programmed to transmit information for ordering dispensing to the remote database when a customized security key is identified by the dispenser.

18. The dispensing system as claimed in claim 15 , wherein the customized security key is an alphanumeric code and the identification device is a physical or digital input keypad arranged on the local interface and connected to the central processing unit.

19. The dispensing system as claimed in claim 15 , wherein the customized security key is carried by a personal object of the user and the identification device is capable of identifying the customized security key and of communicating it to the remote central processing unit (30).

20. The dispensing system as claimed in claim 19, wherein the customized security key is a visual code and the identification device is a scanner connected to the central processing unit.

21. The dispensing system as claimed in claim 19, wherein the customized security key is a biometric feature and the identification device is a corresponding biometric sensor.

22. The dispensing system as claimed in claim 19, wherein the customized security key is a wireless digital signal transmitted by a transmitter carried by the personal object of the user, and the identification device is a wireless receiver connected to the central processing unit for identification.

23. The dispensing system as claimed in claim 22, wherein the wireless digital signal is a proximity radio signal, the local central processing unit (11) or the remote central processing unit (31) being programmed to identify the wireless digital signal, to evaluate the strength of said signal sensed by the wireless receiver (14), and to authorize dispensing of a customized mixture only if the strength of said sensed signal is higher than a threshold strength.

24. The dispensing system as claimed in claim 22, wherein the wireless digital signal is geolocation information relating to the transmitter carried by the personal object of the user, the local automatic dispenser itself comprising a geolocation beacon connected to the local central processing unit and providing geolocation information relating to the dispenser, the local central processing unit (11) or the remote central processing unit (31) being programmed to compare the geolocation information relating to the transmitter carried by the personal object (15a, 15b, 15c) of the user and the geolocation information relating to the dispenser (10), and to authorize dispensing of a customized mixture only if the transmitter carried by the personal object (15a, 15b, 15c) of the user and the dispenser are spaced by a distance less than a threshold distance.

25. The dispensing system as claimed in claim 19 , wherein the personal object of the user is chosen from among a mobile phone, a smartwatch and an object for collecting the customized preparation.
